(19) United States
(12) Patent Application Publication
(10) Pub. No.: US 2026/0201065 A1
(43) Pub. Date: Jul. 16, 2026

(54) USE OF LYSOZYME-C-SPECIFIC ANTIBODY IN PREPARATION OF DRUG FOR TREATING LIVER CANCER

(71) Applicant: ACADEMY OF MILITARY MEDICAL SCIENCES, Beijing (CN)

(72) Inventors: Aihua SUN, Beijing (CN); Fuchu HE, Beijing (CN); Zhiwen GU, Beijing (DN)

(21) Appl. No.: 19/132,726

(22) PCT Filed: Dec. 27, 2023
(86) PCT No.: PCT/CN2023/142279

§ 371 Date: May 23, 2025

(30) Foreign Application Priority Data
Dec. 27, 2022 (CN) 202211681584.4

(51) Int. Cl. C07K 16/40; A61K 39/00; A61P 35/00

(52) U.S. Cl. CPC C07K 16/40; A61P 35/00; A61K 2039/505; C07K 2317/14; C07K 2317/76

(57) ABSTRACT

Disclosed in the present disclosure is the use of a lysozyme C-specific antibody in the preparation of a drug for treating liver cancer, which belongs to the field of medical formulations. The technical problem to be solved by the present application is how to provide a new therapeutic option and basis for hepatocellular carcinoma. In order to solve the technical problem, provided in the present application is the use of a lysozyme C-specific antibody in the preparation of a drug for treating and/or preventing liver cancer. The drug inhibits the proliferation and migration of liver cancer cells and inhibits the growth of hepatic malignant tumors, and the lysozyme C-specific antibody is a monoclonal antibody or polyclonal antibody against lysozyme C. In the study, the effect of the lysozyme C-specific antibody in the treatment of hepatocellular carcinoma is evaluated independently from different angles in vivo and in vitro, so as to provide a new option and basis for the treatment of malignant hepatocellular carcinoma.

The present application claims the right of priority for Chinese patent application no. 202211681584.4, filed with the China National Intellectual Property Administration on Dec. 27, 2022 and entitled “USE OF LYSOZYME C-SPECIFIC ANTIBODY IN THE PREPARATION OF DRUG FOR TREATING LIVER CANCER”, which is incorporated herein by reference in its entirety.

### TECHNICAL FIELD

The present application belongs to the technical field of medical formulations, and particularly relates to the use of a lysozyme C-specific antibody in the preparation of a drug for treating liver cancer.

### BACKGROUND

Hepatocellular carcinoma (HCC) is a major malignant tumor of the liver that can be eradicated at early diagnosis with local therapy, including surgical resection, radiofrequency ablation, transcatheter arterial chemoembolization, or liver transplantation. However, HCC is often diagnosed at an advanced stage, making these radical therapies often ineffective. Therefore, it is particularly urgent to explore new treatment strategies.

Sorafenib tosylate is a new multi-targeted anti-tumor drug successfully developed by Bayer Pharmaceuticals, Germany, which can act on both tumor cells and tumor vessels. It has dual anti-tumor effects: It can inhibit the proliferation of tumor cells directly by blocking the cell signaling pathway mediated by RAF/MEK/ERK, and indirectly inhibit the growth of tumor cells by blocking the formation of tumor neovascularization through the inhibition of vascular endothelial growth factor receptor (VEGFR) and platelet-derived growth factor (PDGF) receptor. It has shown broad anti-tumor activity in preclinical animal experiments and has good tolerability to the treatment, with the main adverse reactions being controllable diarrhea, rash, fatigue, hand-foot syndrome, hypertension, hair loss, nausea/vomiting and loss of appetite. Lysozyme C (LYZ) is secreted mainly by mononuclear macrophages and plays a biological role in the antibacterial infection of the body. In addition, studies have shown that malignant HCC cells may also abnormally express and secrete LYZ, which may be associated with the malignant progression of the tumor. However, there is currently no report in the existing research on whether targeting LYZ can be used as a treatment means for malignant HCC. Therapeutic antibodies have shown great potential for use in the treatment of various diseases such as tumors and autoimmune diseases. However, there is limited research on the use of antibodies in the field of therapy targeting HCC.

### SUMMARY

The technical problem to be solved by the present application is how to provide a new therapeutic option and basis for hepatocellular carcinoma.

In order to solve the technical problem described above, in a first aspect, the present application provides the use of a lysozyme-C-specific antibody in the preparation of a drug for treating and/or preventing liver cancer.

Further, in the use described above, the liver cancer is primary liver cancer.

Further, in the use described above, the liver cancer may be hepatocellular carcinoma.

Further, in the use described above, the drug can inhibit the proliferation of hepatocellular carcinoma cells.

Further, in the use described above, the drug can inhibit the migration of hepatocellular carcinoma cells.

Further, in the use described above, the drug can inhibit the growth of hepatocellular carcinoma.

Further, the hepatocellular carcinoma cells can express lysozyme C (LYZ).

Further, in the use described above, the drug comprises the lysozyme C (LYZ)-specific antibody and a pharmaceutically acceptable carrier material.

Further, in the use described above, the lysozyme C (LYZ)-specific antibody may be a monoclonal antibody or a polyclonal antibody against lysozyme C.

Further, in the use described above, the lysozyme C (LYZ)-specific antibody may be a polyclonal antibody against lysozyme C.

In the present application, the carrier material includes, but is not limited to, water-soluble carrier materials (such as polyethylene glycol, polyvinylpyrrolidone, and organic acids), poorly soluble carrier materials (such as ethylcellulose and cholesterol stearate), and enteric-soluble carrier materials (such as cellulose acetate phthalate and carboxymethyl ethyl cellulose). These materials can be used to prepare various dosage forms, including but not limited to tablets, capsules, drop pills, aerosols, pills, powders, solutions, suspensions, emulsions, granules, liposomes, transdermal agents, buccal tablets, suppositories, lyophilized powders, etc. These dosage forms may be conventional preparations, sustained-release preparations, controlled-release preparations, and various particulate drug delivery systems. In order to prepare the unit dosage form into tablets, various carriers well known in the art can be widely used. Examples regarding carriers are, for example, diluents and absorbents, such as starch, dextrin, calcium sulfate, lactose, mannitol, sucrose, sodium chloride, glucose, urea, calcium carbonate, kaolin, microcrystalline cellulose, aluminum silicate, etc.; wetting agents and binders, such as water, glycerol, polyethylene glycol, ethanol, propanol, starch paste, dextrin, syrup, honey, glucose solution, acacia mucilage, gelatin paste, sodium carboxymethylcellulose, shellac, methylcellulose, potassium phosphate, polyvinylpyrrolidone, etc.; disintegrants, for example, dried starch, alginate, agar powder, laminaran, sodium bicarbonate and citric acid, calcium carbonate, polyoxyethylene, sorbitan fatty acid esters, sodium dodecyl sulfonate, methylcellulose, ethylcellulose, etc.; disintegration inhibitors, for example, sucrose, glyceryl tristearate, cocoa butter, hydrogenated oil, etc.; absorption promoters, for example, quaternary ammonium salts, sodium dodecyl sulfate, etc.; lubricants, for example, talc, silica, corn starch, stearate, boric acid, liquid paraffin, polyethylene glycol, etc. Tablets may also be further prepared into coated tablets, for example, sugar-coated tablets, film-coated tablets, enteric-coated tablets, or double-layer tablets and multi-layer tablets. In order to prepare the unit dosage form into pills, various carriers well known in the art can be widely used. Examples regarding carriers are, for example, diluents and absorbents, such as glucose, lactose, starch, cocoa butter, hydrogenated vegetable oil, polyvinylpyrrolidone, kaolin, talc, etc.; binders, such as acacia, tragacanth, gelatin, ethanol, honey, liquid sugar, rice paste or batter, etc.; disintegrants, such as agar powder, dried starch, alginate, sodium dodecyl sulfate, methyl cellulose, ethyl cellulose, etc. In order to prepare the unit dosage form into suppositories, various carriers well known in the art can be widely used. Examples regarding carriers are, for example, polyethylene glycol, lecithin, cocoa butter, higher alcohols, esters of higher alcohols, gelatin, semi-synthetic glycerides, etc. In order to prepare the unit dosage form into preparations for injection, such as solutions, emulsions, lyophilized powders and suspensions, all diluents commonly used in the art can be used, for example, water, ethanol, polyethylene glycol, 1,3-propanediol, ethoxylated isostearyl alcohol, polyoxylated isostearyl alcohol, polyoxyethylene sorbitol fatty acid esters, etc. In addition, in order to prepare isotonic injection solutions, an appropriate amount of sodium chloride, glucose or glycerol can be added to the preparations for injection. In addition, conventional cosolvents, buffers, pH regulators, etc., can be added. In addition, colorants, preservatives, perfuming agents, flavoring agents, sweetening agents or other materials can be added to the pharmaceutical preparations.

The dosage forms described above can be administered by injection, including subcutaneous injection, intravenous injection, intramuscular injection, intraluminal injection, etc. The dosage forms described above can also be administered orally.

The beneficial technical effects achieved by the present application are as follows:

In order to explore the feasibility of LYZ-specific antibodies in the treatment of hepatocellular carcinoma, in the study, the effect of the lysozyme C (LYZ)-specific antibody in the treatment of hepatocellular carcinoma is evaluated independently from different angles in vivo and in vitro, so as to provide a new option and basis for the treatment of malignant hepatocellular carcinoma.

### BRIEF DESCRIPTION OF THE DRAWINGS

FIG. 1 shows the detection results of the expression levels of LYZ in different HCC cells. IB: α-LYZ in FIG. 1 represents the expression levels of LYZ in different HCC cells. IB: α-LYZ (S) in FIG. 1 represents the secretion levels of LYZ in different HCC cells. IB: α-β-Actin in FIG. 1 represents the expression levels of the internal reference protein Actin in different HCC cells.

FIG. 2 shows the experimental results of LYZ-specific antibodies inhibiting the in-vitro proliferation of hepatocellular carcinoma cells in a concentration-dependent manner. HepG2, PLC/PRF/5 and SNU-387 cells are respectively treated with InVivoMab polyclonal rabbit IgG (Ctrl) and different concentrations of rabbit anti-human LYZ polyclonal antibodies (Ab5: 5 μg/mL, Ab10: 10 μg/mL). The proliferation of the cells under different treatments is continuously monitored by the xCELLigence RTCA DP real-time cell detection system. The experiment is independently repeated 3 times, and the data are presented in the form of (mean±standard error); *** indicates P<0.001, and n.s. indicates no statistical difference.

FIG. 3 shows the experimental results of LYZ-specific antibodies inhibiting the in-vitro migration of hepatocellular carcinoma cells in a concentration-dependent manner. HepG2 cells (A) or Huh-7 cells (B) are respectively treated with InVivoMab polyclonal rabbit IgG (control IgG) or different concentrations of rabbit anti-human LYZ polyclonal antibodies (Ab5: 5 μg/mL, Ab10: 10 μg/mL, Ab20: 20 μg/mL). The influence of treatment with different concentrations of antibodies on the migration of hepatocellular carcinoma cells is detected by the Transwell technique. At least 5 high-power fields are counted under a microscope. The experiment is independently repeated 3 times, and the data are presented in the form of (mean±standard error). *** indicates P<0.001. The scale bar in FIG. A represents 200 μm, and the scale bar in FIG. B represents 100 μm.

FIG. 4 shows the experimental results of the inhibition of the growth of hepatocellular carcinoma in mice by the LYZ-specific antibody. Different groups of NOD/SCID tumor-bearing mice are respectively treated with InVivoMab polyclonal rabbit IgG (control IgG, 200 μg/mouse), rabbit anti-human LYZ polyclonal antibody (LYZ-specific antibody, 200 μg/mouse), and sorafenib tosylate (15 mg/kg). The differences in tumor growth in different groups of mice are monitored by the in-vivo imaging technique. The data are presented in the form of (mean±standard error), * indicates P<0.05, and ** indicates P<0.01.

### DETAILED DESCRIPTION OF THE EMBODIMENTS

The present application is further described in detail below in conjunction with detailed description, and the examples given are only used to illustrate the present application, but are not to limit the scope of the present application. The examples provided below may serve as guidelines for further improvements by one of ordinary skill in the art and do not limit the present application in any way.

Unless otherwise specified, the experimental methods in the following examples are conventional methods, and are performed according to techniques or conditions described in the literatures in the art or according to the product instructions. The materials, reagents, etc., used in the following examples are all commercially available, unless otherwise specified.

The main experimental materials and sources of the present application were as follows:

The human hepatocellular carcinoma cell line HepG2 was purchased from the Cell Bank of Type Culture Collection Committee of the Chinese Academy of Sciences under catalog number SCSP-510. The human hepatocellular carcinoma cell line Huh-7 was purchased from the Cell Bank of Type Culture Collection Committee of the Chinese Academy of Sciences under catalog number SCSP-526. The human hepatocellular carcinoma cell line Hep3B was purchased from the Cell Bank of Type Culture Collection Committee of the Chinese Academy of Sciences under catalog number SCSP-5045. The human hepatocellular carcinoma cell line PLC/PRF/5 was purchased from the Cell Bank of Type Culture Collection Committee of the Chinese Academy of Sciences under catalog number SCSP-5095. The human hepatocellular carcinoma cell line SNU-387 was purchased from the Cell Bank of Type Culture Collection Committee of the Chinese Academy of Sciences under catalog number SCSP-5046. The human embryonic kidney cell line 293T was purchased from the Cell Bank of Type Culture Collection Committee of the Chinese Academy of Sciences under catalog number SCSP-502. The human hepatocellular carcinoma cell line (SNU-475) was purchased from Shanghai Zhong Qiao Xin Zhou Biotechnology Co., Ltd. under the product number ZQ0706.

Huh-7 cells (Huh-7-Luc) expressing the luciferase reporter gene were constructed by our laboratory. A target plasmid (pGL4.51[luc2/CMV/Neo], purchased from Promega, catalog number: E132A) was transfected into Huh-7 cells using a Lipofectamine™ 3000 transfection reagent (purchased from Thermo Fisher Scientific, catalog number: L3000015), and 48 hours after transfection, Huh-7 cells stably expressing the luciferase reporter gene were selected using a G418 selective antibiotic (purchased from Thermo Fisher Scientific, catalog number: 10131035, used at a concentration of 1 mg/mL) as target cells.

HepG2, Huh-7/Huh-7-Luc, Hep3B and PLC/PRF/5 cells were cultured in a DMEM basal medium (Gibco, C11995500BT) containing 10% fetal bovine serum (Newzerum, FBS-S500) and 1% double antibiotics solution (Gibco, 15140-122), and SNU-387 and SNU-475 cells were cultured in an RPMI 1640 basal medium (Gibco, C11875500BT) containing 10% fetal bovine serum (Newzerum, FBS-S500) and 1% double antibiotics solution (Gibco, 15140-122). The cells were passaged once every two days. After being digested with trypsin (Gibco, 25200-056), the cells were placed in a cell incubator (Thermo, Heracell VIOS 250i) at 37° C. with 5% CO₂ for culture. All cells were verified by STR detection and were free of mycoplasma contamination.

Main reagents: Rabbit anti-human LYZ polyclonal antibody (Agilent, A0099), InVivoMab polyclonal rabbit IgG (BioXCell, BE0095), sorafenib tosylate (APExBIO, A8245), rabbit anti-LYZ monoclonal antibody (Abcam, ab108508), mouse anti-3-Actin antibody (Proteintech, 66009-1-Ig), HRP-conjugated goat anti-rabbit IgG (Proteintech, SA00001-2), HRP-conjugated goat anti-mouse IgG (Proteintech, SA00001-1), cell protein extraction reagent (Thermo, 89901), BCA protein quantification reagent (Thermo, 23225), crystal violet staining solution (Beyotime, C0121), Matrigel matrix (Corning, 356237), and D-luciferin potassium salt (Beyotime, ST196).

Main instruments and consumables: Protein electrophoresis and transfer apparatus for Western blot (BioRad, 1658033), real-time cell analysis system (Agilent, xCELLigence RTCA DP), benchtop high-speed refrigerated centrifuge (Eppendorf, 5425R), E-Plate 16 microplate (Agilent, 5469830001), 8 μm Boyden chamber (FALCON, 353097), 10 kD protein concentration ultrafiltration tube (Millipore, UFC501096), and PVDF protein transfer membrane (BioRad, 1620184).

Male NOD/SCID mice aged 6-8 weeks used in the experiment were purchased from Beijing Vital River Laboratory Animal Technology Co., Ltd. The mice were housed in the SPF animal facility of the National Center for Protein Science (Beijing). The animal experiments involved in the present application passed the review of the Institutional Animal Care and Use Committee of the National Center for Protein Science (Beijing). Ethical review number: IACUC-20210329-16MT.

In the following examples, the data were processed and analyzed using GraphPad Prism 7.00 software. The results were expressed as mean±standard deviation. One-way ANOVA or Two-way ANOVA tests were used. * indicates a significant difference (P<0.05), ** indicates an extremely significant difference (P<0.01), *** indicates an extremely significant difference (P<0.001), and n.s. indicates no statistical difference.

In the present application, the expression and secretion of LYZ in human hepatocellular carcinoma cell lines were detected by Western blot. The influence of LYZ-specific antibodies on the in-vitro proliferation of HCC cells was detected by the real-time cell analysis system. The influence of LYZ-specific antibodies on the in-vitro migration of HCC cells was detected by the Transwell technique. The influence of LYZ-specific antibodies on the in-vivo growth of HCC tumors was detected using an orthotopic liver transplantation tumor model in mouse.

### Example 1: High Expression of LYZ in Some HCC Cells

HCC cells cultured for two days were collected, including a total of six human hepatocellular carcinoma cells, namely HepG2, Huh-7, Hep3B, PLC/PRF/5, SNU-387, and SNU-475. The total proteins of the six human hepatocellular carcinoma cell lines were extracted according to the product instructions of a cell protein extraction reagent. Then, the concentrations of various protein samples were detected using a BCA protein quantification reagent, and 30 μg of each protein was taken for Western blot analysis to detect the expression levels of LYZ in the six human hepatocellular carcinoma cells. Meanwhile, 500 μL of each of the cell culture supernatants was taken and added to a 10 kD protein concentration ultrafiltration tube. The protein concentration ultrafiltration tube was placed in a centrifuge and centrifuged at 1000 rpm at 4° C. When the supernatant was concentrated until a volume of about 50 μL remained, 2 μL of the concentrated supernatant was taken for Western blot (WB) analysis to detect the secretion levels of LYZ in the six human hepatocellular carcinoma cell lines.

The expression levels of proteins were determined by Western blot. During electrophoresis, the voltage was first set at 80 V, and after the samples migrated into the separating gel, the voltage was adjusted to 150 V until the samples completely migrated to the bottom of the separating gel. Then, the conditions (90 V, 90 min) were set to transfer the protein bands in the separating gel onto a PVDF membrane, and the expression and secretion levels of LYZ in various cells were determined using rabbit anti-LYZ monoclonal antibody and mouse anti-β-Actin antibody.

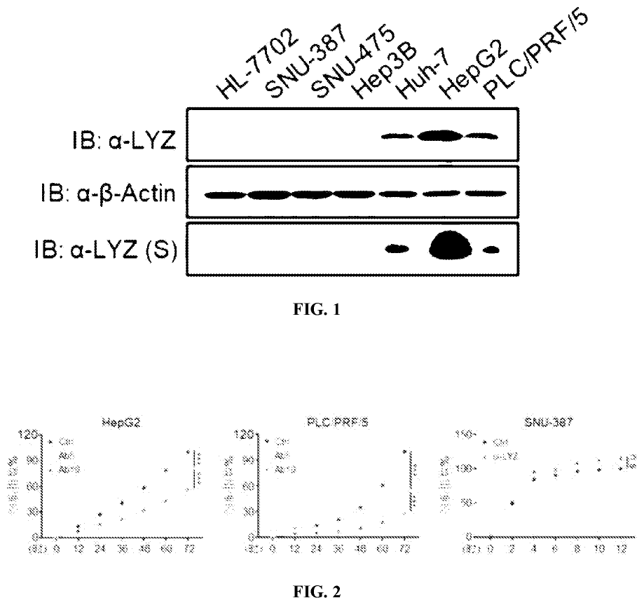

The results are as shown in FIG. 1. IB: α-LYZ in FIG. 1 represents the Western blot detection results of the expression levels of LYZ in six human hepatocellular carcinoma cells. IB: α-β-Actin in FIG. 1 represents the Western blot detection results of the expression levels of β-Actin in six human hepatocellular carcinoma cell lines. IB: α-LYZ (S) in FIG. 1 represents the Western blot detection results of the secretion levels of LYZ in six human hepatocellular carcinoma cell lines. The results showed that the expression and secretion levels of LYZ were the highest in HepG2 cells, followed by those in Huh-7 cells and PLC/PRF/5 cells, and no LYZ expression was detected in SNU-387 and SNU-475. Therefore, HepG2, Huh-7 and PLC/PRF/5 cells were selected as experimental cells for the subsequent evaluation of the therapeutic effects of LYZ-specific antibodies.

### Example 2: Inhibition of In-Vitro Proliferation of HCC Cells by LYZ-Specific Antibodies in a Concentration-Dependent Manner

HepG2, PLC/PRF/5 and SUN-387 cells cultured for two days were collected and counted. According to 8000/well×100 μL for HepG2, 5000/well×100 μL for PLC/PRF/5, and 5000/well×100 μL for SNU-387, the three human hepatocellular carcinoma cells (HepG2, PLC/PRF/5, SNU-387) were seeded and grouped as follows with three replicate wells set for each experimental group.
Group 1: Control IgG treatment group, in which the InVivoMab polyclonal rabbit IgG was added to the human hepatocellular carcinoma cell suspension;
Group 2: 5 μg/mL LYZ-specific antibody treatment group, in which the rabbit anti-human LYZ polyclonal antibody was added to the human hepatocellular carcinoma cell suspension (final concentration of the antibody in the system was 5 μg/mL);
Group 3: 10 μg/mL LYZ-specific antibody treatment group, in which the rabbit anti-human LYZ polyclonal antibody was added to the human hepatocellular carcinoma cell suspension (final concentration of the antibody in the system was 10 μg/mL).

The culture medium of HepG2, PLC/PRF/5 or SNU-387 cells was taken and centrifuged at 800 rpm for 3 minutes. Then, the supernatant was discarded and the cells were resuspended in the corresponding volume of DMEM or RPMI 1640 μmedium containing 2% FBS. According to the experimental groups, total number of cells required for various groups was calculated, and then the InVivoMab polyclonal rabbit IgG or different concentrations of rabbit anti-human LYZ polyclonal antibodies (the final concentration of the antibody in the system was 5 μg/mL or 10 μg/mL) were added to the cell suspension of various experimental groups. After thorough mixing, cells in various groups were added to an E-Plate 16 microplate (100 μL/well) and the differences in the cell proliferation of different groups were monitored by the xCELLigence RTCA DP real-time cell analysis system.

The results are as shown in FIG. 2. The abscissa in FIG. 2 represents the culture time of cells in the E-Plate 16 microplate, and the ordinate in FIG. 2 represents the cell index calculated by the instrument for the proliferation of various cells in the E-Plate 16 microplate. The cell index is positively correlated with cell proliferation. The results of FIG. 2 showed that HepG2, PLC/PRF/5 and SNU-387 cell index were increasing as the culture time increased. Importantly, rabbit anti-human LYZ polyclonal antibody treatment significantly inhibited the proliferation of two hepatocellular carcinoma cells, HepG2 and PLC/PRF/5, and exhibited a significant concentration-dependent trend. However, it did not inhibit the proliferation of SNU-387 cells that did not express LYZ themselves, indicating that LYZ-specific antibodies had the effect of inhibiting the in-vitro proliferation of LYZ-expressing hepatocellular carcinoma cells.

### Example 3: Inhibition of In-Vitro Migration of HCC Cells by LYZ-Specific Antibodies in a Concentration-Dependent Manner

The experimental groups for the two human hepatocellular carcinoma cells (HepG2, Huh-7) were as follows:
Group 1: Control IgG treatment group, in which the InVivoMab polyclonal rabbit IgG was added to the human hepatocellular carcinoma cell suspension;
Group 2: 5 μg/mL LYZ-specific antibody treatment group, in which the rabbit anti-human LYZ polyclonal antibody was added to the human hepatocellular carcinoma cell suspension (final concentration of the antibody in the system was 5 μg/mL);
Group 3: 10 μg/mL LYZ-specific antibody treatment group, in which the rabbit anti-human LYZ polyclonal antibody was added to the human hepatocellular carcinoma cell suspension (final concentration of the antibody in the system was 10 μg/mL);
Group 4: 20 μg/mL LYZ-specific antibody treatment group, in which the rabbit anti-human LYZ polyclonal antibody was added to the human hepatocellular carcinoma cell suspension (final concentration of the antibody in the system was 20 μg/mL).

The cultured HepG2 or Huh-7 cells were collected and counted. According to the experimental system of 4×10⁵/well×200 μL (HepG2) or 1×10⁵/well×200 μL (Huh-7), the cells were resuspended in a DMEM basal medium. Then, the InVivoMab polyclonal rabbit IgG or different concentrations of rabbit anti-human LYZ polyclonal antibodies as shown in the figure were added to the cell suspensions of various experimental groups and mixed thoroughly. 600 μL of DMEM medium containing 20% FBS was added to a 24-well plate, then the 8 μm Boyden chambers were placed into the 24-well plate containing the medium at an inclined angle, and the cell suspensions of the experimental groups were added to the upper chamber of the Boyden chambers and cultured in the incubator for 36 h. The number of cells migrated through the chamber in each experimental group was counted by crystal violet staining. The results are as shown in FIG. 3. The number of HCC cells migrated through the Boyden chamber decreased successively as the concentration of rabbit anti-human LYZ polyclonal antibody increased, indicating that LYZ-specific antibodies could inhibit the in-vitro migration of hepatocellular carcinoma cells.

### Example 4: Inhibition of the In-Vivo Growth of Orthotopic Liver Transplantation Tumor in Mouse by the LYZ-Specific Antibody

The cultured Huh-7-Luc cells were collected and counted, and the cell concentration was adjusted to 0.7×10⁸/mL. The cell suspension with the adjusted concentration was mixed with Matrigel matrix gel at a volume ratio of 1:1. Huh-7-Luc cells were inoculated under the liver capsule of NOD/SCID mice at a volume of 20 μL/mouse. The mice were surgically sutured and fed with 1% double antibiotics for at least 3 days. One week after tumor inoculation, the tumor-bearing mice were randomly divided into three groups as follows according to the in-vivo imaging results, with each group containing 8-10 mice.
Group 1: Control IgG treatment group (10 μmice). Each mouse was treated with InVivoMab polyclonal rabbit IgG, administered at a dose of 200 μg/mouse, in a volume of 100 μL.
Group 2: LYZ-specific antibody treatment group (10 μmice). Each mouse was treated with rabbit anti-human LYZ polyclonal antibody, administered at a dose of 200 μg/mouse, in a volume of 100 μL.
Group 3: Sorafenib tosylate drug treatment group (8 mice). Each mouse was treated with sorafenib tosylate, administered at a dose of 15 mg/kg, in a volume of 100 μL.

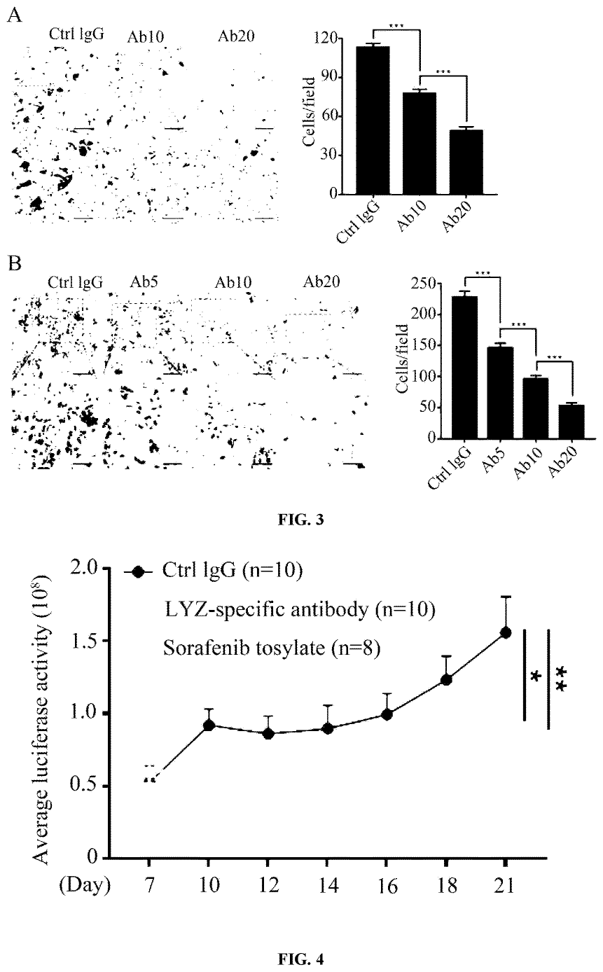

Then, the tumor-bearing mice were administered intraperitoneally daily for two consecutive weeks. Meanwhile, in-vivo imaging was performed three times a week to monitor the tumor growth in the mice of different treatment groups (the mean luciferase activity of tumor cells is positively correlated with tumor size). The results are as shown in FIG. 4. The tumors in mice treated with control IgG continued to grow, whereas treatment with the rabbit anti-human LYZ polyclonal antibody significantly inhibited the tumor growth in mice, and could essentially achieved an equivalent therapeutic effect of the clinically positive drug sorafenib tosylate, indicating that the LYZ-specific antibody could inhibit the in-vivo growth of hepatocellular carcinoma cells in mice.

Combined with the experimental results described above, it is indicated that the LYZ-specific antibody can significantly inhibit the growth of HCC cells both in vitro and in vivo, which may provide potential application and clinical translation value for the treatment of malignant HCC.

The present application has been described in detail above. For a person skilled in the art, the present application can be performed within a wide scope under equivalent parameters, concentrations and conditions without departing from the spirit and scope of the present application and without unnecessary experimentation. Although the present application provides specific examples, it should be understood that further improvements may be made to the present application. In summary, in accordance with the principles of the present application, the present application is intended to include any modification, use or improvement to the present application, including changes departing from the scope disclosed in the present application and using conventional techniques known in the art.

1. A method for treating and/or preventing liver cancer, comprising administrating a lysozyme C-specific antibody to a subject in need.

2. The method according to claim 1, wherein the liver cancer is primary liver cancer.

3. The method according to claim 1, wherein the liver cancer is hepatocellular carcinoma.

4. The method according to claim 1, wherein the lysozyme C-specific antibody is a monoclonal antibody against lysozyme C or a polyclonal antibody against lysozyme C.

5. The method according to claim 1, wherein the lysozyme C-specific antibody inhibits the proliferation of hepatocellular carcinoma cells.

6. The method according to claim 5, wherein the hepatocellular carcinoma cells are HepG2 cells or PLC/PRF/5 cells.

7. The method according to claim 1, wherein the lysozyme C-specific antibody inhibits the migration of hepatocellular carcinoma cells.

8. The method according to claim 7, wherein the hepatocellular carcinoma cells are HepG2 cells or Huh-7 cells.

9. The method according to claim 1, wherein the lysozyme C-specific antibody inhibits the growth of hepatocellular carcinoma cells.

10. The method according to claim 9, wherein the hepatocellular carcinoma cells are Huh-7-Luc cells.

11. The method according to claim 1, wherein the lysozyme C-specific antibody is in the form of a drug, the drug comprises the lysozyme C-specific antibody and a pharmaceutically acceptable carrier.

12. The method according to claim 11, wherein the lysozyme C-specific antibody is a monoclonal antibody against lysozyme C or a polyclonal antibody against lysozyme C.

13. The method according to claim 11, wherein the pharmaceutically acceptable carrier is a water-soluble carrier material, a poorly soluble carrier material or an enteric-soluble carrier material.

14. The method according to claim 11, wherein the dosage form is a tablet, a capsule, a drop pill, an aerosol, a pill, a powder, a solution, a suspension, an emulsion, a granule, a liposome, a transdermal agent, a buccal tablet, a suppository or a lyophilized powder.

15. The method of claim 4, wherein the liver cancer is primary liver cancer.

16. The method of claim 4, wherein the liver cancer is hepatocellular carcinoma.

17. The method of claim 5, wherein the liver cancer is primary liver cancer.

18. The method of claim 7, wherein the liver cancer is primary liver cancer.

19. The method of claim 9, wherein the liver cancer is primary liver cancer.

20. The method of claim 11, wherein the liver cancer is primary liver cancer.
